(19) United States
(12) Patent Application Publication
(10) Pub. No.: US 2013/0319405 A1
(43) Pub. Date: Dec. 5, 2013

(54) LOCKING DEVICE FOR AN ENDOSTOMAL TRACHEOSTOMA BUTTON

(71) Applicant: Robert G. Perry, Indianapolis, IN (US)

(72) Inventor: Robert G. Perry, Indianapolis, IN (US)

(21) Appl. No.: 13/486,448

(22) Filed: Jun. 1, 2012

(51) Int. Cl. A61M 16/04

(52) U.S. Cl. 128/200.26

(57) ABSTRACT

A locking device is disclosed for use with a resilient flexible endostomal tracheostoma button. Commonly used trachostoma buttons include a retaining lip situated at one end of a cylindrical portion that is insertable into the stoma of the patient and serves to retain the button in position in the stoma. The locking device invention is a hollow cylindrical tube that is removably inserted into the tracheostoma button after insertion of the tracheostoma button into the patient's stoma. One end of the locking device includes an insertion stop radial protrusion for proper positioning of the locking device within the button. The locking device internally supports the walls and retaining lip of the tracheostoma button and substantially increases the retaining force of the lip, thereby reducing awkward ejection of the tracheostoma tube, such as during patient coughing.

### FIELD OF THE INVENTION

This invention relates in general to prosthesis devices and more particularly to endostomal tracheostoma tubes or buttons.

### BACKGROUND OF THE INVENTION

Medical procedures that result in tracheoesophageal puncture create a need for reliable medical devices such as endostomal tracheostoma buttons. Such procedures include, but are not limited to, laryngectomy, tracheostomy and throat cancer operations. After such operations, the patient will have a stoma in the lower throat area through which breathing is achieved. It is common for a cannula to be inserted in the stoma in order to keep the passageway open for breathing. If left alone, the stoma will close naturally by way of the healing process. A permanent stoma is created surgically using circumferential sutures.

A number of cannula devices are known in the art that are insertable into a stomal opening to aid in breathing and adapted for use with tracheostoma valves. One of the more well known devices is the Barton-Mayo™ button, an endostomal tracheostoma tube widely used by many tracheostomy patients. The Barton-Mayo™ button is used in conjunction with tracheostoma valves, such as the device shown in U.S. Pat. No. 5,059,208, to enable speech for patients whose larynx has been removed. Another well known similar device is manufactured by Atos Medical Inc. and referred to as the Provox LaryButton™. The Larybutton device is a short, self-retaining silicone tracheal cannula. The LaryButton™ device maintains the opening of the tracheostoma and acts as a holder for other devices such as tracheostoma valves.

It is known in the art to use tracheostoma valves with a movable diaphragm as shown in the 5,059.208 patent. The diaphragm is normally positioned in an open position. During normal breathing, pressures are insufficient to move the diaphragm to a closed position. Thus, the patient will inhale and exhale past the diaphragm. Air pressures for speech purposes are initiated at somewhat higher levels. These higher pressures move the diaphragm to a closed position, blocking the discharge of air to the atmosphere. The exhaled air is then diverted either through the esophogas for esophageal speech or through a voice prostheses device to the oral cavity to produce speech.

The external radius of a tracheostoma button must correspond closely with the patient's stoma for a proper air seal and retention within the stoma. Various diameter sizes and tube lengths for tracheostoma buttons are provided by manufacturers, however, the possibility of awkward and embarrassing forceful ejection of some tracheostoma buttons may still occur if the patient coughs or breathes too strongly due to the collapsible nature of the button's material. Further, custom cross-section designed tracheostoma buttons for patients whose stoma is irregular or non-circular are costly to produce and impose a financial burden on patients whose stoma is not substantially circular in geometry. Where the stoma opening is irregular or non-circular, a device that urges the cylindrical portion of the button in contact with the stoma would improve the air seal achieved.

Another consideration for tracheostomy patients is the natural expansion of the stoma. Overnight, when the tube is removed, the stoma contracts slightly. When the tube is inserted into the stoma in the morning, a gradual enlargement of the stoma occurs while the tube is in place that degrades the “fit” of the tube in the stoma resulting in air leaking past the tube.

It may also be desirable for some patients to achieve an enlargement of the stoma to increase the opening size and allow more air to readily pass through. Where the stoma is larger, a larger diameter tracheostoma button is used by the patent. Just as a skin piercing may be enlarged by the insertion of larger objects through the pierced flesh, it may be desirable for a tracheostomy patent to achieve an enlarged stoma. An inexpensive and simple device to achieve such results is needed.

What is needed is an improvement over the prior art to more securely retain certain tracheostoma buttons in a patient's stoma under increased air pressure conditions to prevent undesirable ejection of the button as a result of coughing or the like. Where the patient's stoma has expanded during daily usage of a tracheostoma button, a device used in conjunction with the button that improves the fit of the button without the requirement to replace the button with a larger button is needed. Where stoma enlargement is desired to improve breathing, a device to achieve such without surgery is also needed.

### SUMMARY OF THE INVENTION

A locking device for use with an endostomal tracheostoma button, wherein the endostomal tracheostoma button is fabricated from a resilient flexible material and includes a hollow cylindrical portion disposed into a patient's stoma, and wherein the tracheostoma button includes a lip situated at one end of the cylindrical portion that serves to retain the tracheostoma button within the stoma aperture, the locking device according to one aspect of the present invention comprises a hollow cylinder, the hollow cylinder having a first end and a second end, and wherein the radius of the hollow cylinder is greater than the internal radius of the hollow cylindrical portion of the endostomal tracheostoma button; and means attached to the first end of the hollow cylinder for limiting insertion depth when the second end of the hollow cylinder is axially inserted into the hollow cylindrical portion of the endostomal tracheostoma button, the means for limiting positioning the second end of the hollow cylinder radially adjacent the lip of the tracheostoma button when the hollow cylinder is fully inserted into the hollow cylindrical portion of the tracheostoma button, and wherein the second end of the hollow cylinder provides outward radial support for the lip of the tracheostoma button.

One object of the present invention is to provide a more reliable tracheostoma button.

Another object of the present invention is to reduce the costs associated with fabrication of custom designed tracheostoma buttons.

Still another object of the present invention is to ensure a secure installation of a tracheostoma button in a patient's stoma.

Yet another object of the present invention is to improve the patient experience by reducing the likelihood of an unexpected ejection of a tracheostoma button from a patient's stoma.

A further object of the present invention is to provide a non-surgical mechanism for enlarging the patient's stoma to improve breathing capability.

These and other objects of the present invention will become more apparent from the following description of the preferred embodiments.

### BRIEF DESCRIPTION OF THE DRAWINGS

FIG. 1 is a cross-sectional view of a prior art Barton-Mayo™ tracheostoma button inserted into a stoma.

FIG. 2 is a side view of the Barton-Mayo™ tracheostoma button.

FIG. 3 is an end view of the Barton-Mayo™ tracheostoma button.

FIG. 4 is a cross-sectional view of a locking device according to one aspect of the present invention installed in a Barton-Mayo™ tracheostoma button.

FIG. 5 is a front view of the locking device of FIG. 4.

FIG. 6 is a rear view of the locking device of FIG. 4.

FIG. 7 is a side view of the locking device of FIG. 4.

### DESCRIPTION OF THE PREFERRED EMBODIMENTS

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

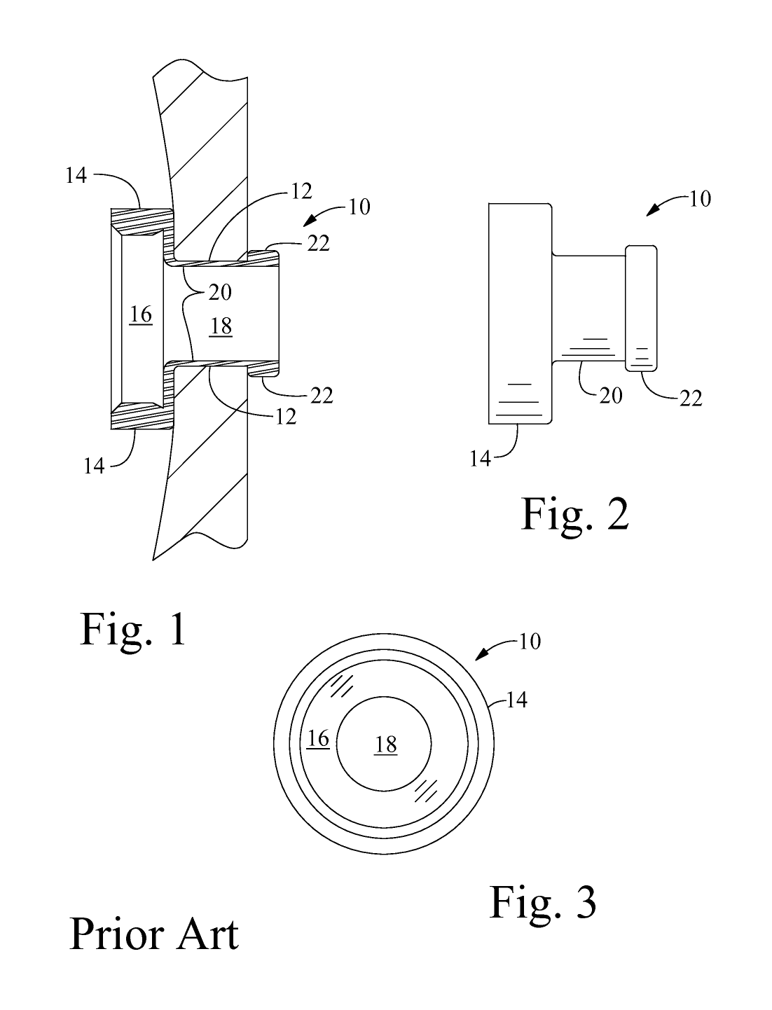

Referring now to FIGS. 1-3, a prior art Barton-Mayo™ tracheostoma tube or button 10 is shown. The Barton-Mayo™ tracheostoma tube is commonly referred to in the industry as a tracheostoma “button” and will be referred to herein as such. FIG. 1 is a cross-sectional drawing of a patient's throat area depicting a Barton-Mayo™ button 10 disposed in a patient's stoma 12. Typically, stoma 12 is located in the lower throat area of the neck below the original larynx location. FIG. 2 is a side view of button 10 and FIG. 3 is a valve end view of button 10. Button 10 includes a larger diameter hollow cylindrical portion 14 that defines a cylindrical aperture 16 within which a tracheostoma valve (not shown) is removably inserted. A smaller diameter cylindrical aperture 18 is defined by the smaller diameter cylindrical portion 20 of button 10. Button 10 also includes retaining protrusion or lip 22. Button 10 is constructed of a semi-rigid silicone material that is resilient and flexible yet capable of being sterilized.

The process for installing button 10 into stoma 12 requires the user to compress the cylindrical portion 20 nearly flat then fold over lip 22 along the cylindrical axis to reduce the size of cylindrical portion 20 and lip 22 for insertion into stoma 12. Upon releasing the folded button 10 in the stoma, cylindrical portion 20 springs back to the cylindrical shape shown, with the outer wall of cylindrical portion 20 contacting the surfaces of stoma 12. Lip 22 resiliently expands to its original shape and is situated within the patient's body and functions to retain button 10 in position within stoma 12. The diameter and length of cylindrical portion 20 are patient specific, thus button 10 is manufactured and sold in a number of size variations for these two dimensional attributes. The external lateral surface of cylindrical portion 20 physically contacts the stoma and achieves an air tight seal with stoma 12 when the diameter of cylinder 20 is sized appropriately since the material used to construct button 10 is resilient and flexible in nature.

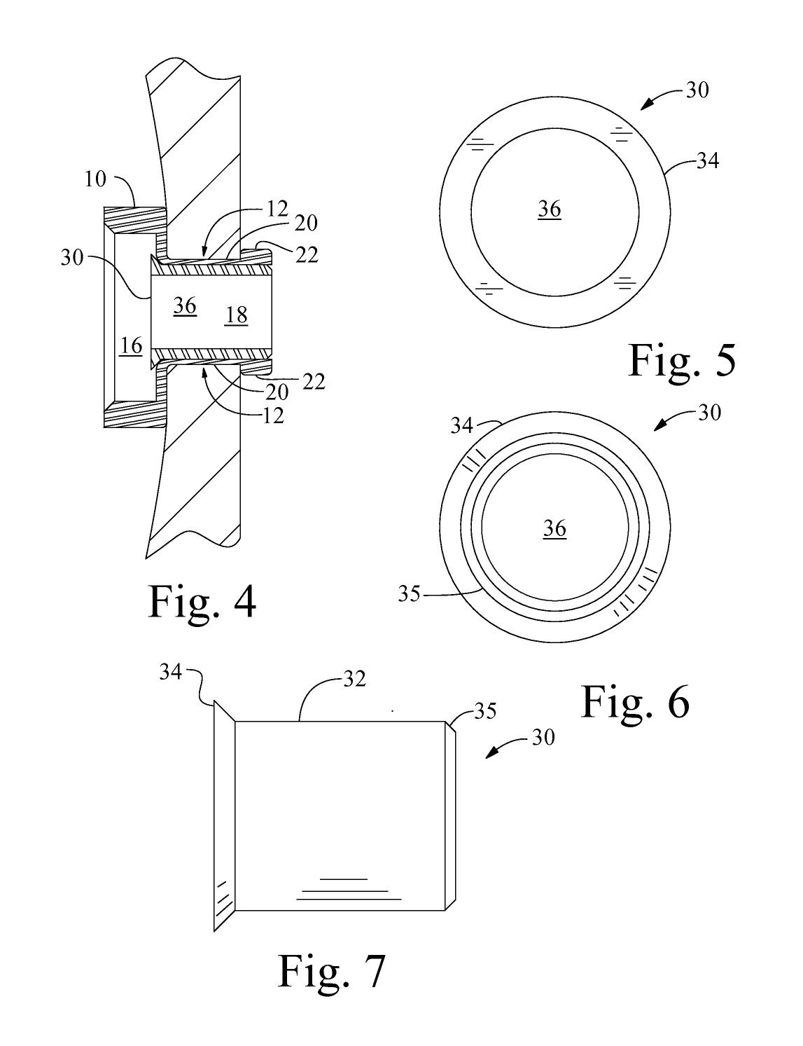

Referring now to FIGS. 4-7, a locking device 30, according to the present invention, for use with a tracheostoma button 10 is shown. Locking device 30 includes hollow cylindrical portion 32 having a radial protrusion 34 situated at one end thereof. In the instant case, protrusion is a radial protrusion whose thickness is reduced radially at approximately a forty-five degree angle to a fairly thin protrusion at the radial maximum. Cylindrical portion 32 includes a through hole or aperture 36 through which patient breathing is achieved. A chamfer or taper 35 is formed in portion 32 to aid in the insertion of device 30 into aperture 18 of button 10. Radial protrusion 34 has a triangular cross-section which enables the user to grasp the outer edge thereof with fingernails or a small tool for easy removal of device 30 from within button 10.

Locking device 30 is axially inserted into button 10 after installation of button 10 in stoma 12. The diameter of protrusion 34 is sized so that device 30 cannot be inserted through aperture 18 yet a trachestoma valve may still be normally received in button 10. When fully inserted into button 10, one end of cylindrical portion 32 is positioned adjacent and radially beneath lip 22 as shown in FIG. 4. The internal radial support of device 30 prevents lip 22 from collapsing and serves to lock button 10 in position in stoma 12. Locking device 30 provides internal radial support for the thin flexible yet resilient walls of cylindrical portion 20 of button 10 maintaining walls 20 and lip 22 in radial position and thereby preventing the undesirable inward collapse of lip 22 which results in undesirable removal of button 10 from within stoma 12, such as during a patient's coughing fit or during extremely heavy breathing from the patient's physical activity. Locking device 30 also provides an improved air seal between the surfaces of stoma 12 and cylindrical portion 20 of button 10.

Locking device 30 is preferably constructed of food grade plastic material or other suitable synthetic materials and may be semi-rigid or rigid in physical properties. Any material that is suitable for machining and sterilization yet will retain a semi-rigid and fixed shape may be used. Device 30 may be machined, formed or molded, whichever is more cost effective. It is also contemplated that protrusion 34, which provides an insertion depth stop means for device 30, may be fabricated in a number of ways, including but not limited to radially extending spokes or teeth, or a fixed width radial protrusion with no taper that extends outward toward the diameter of aperture 16 yet provides clearance for insertion of a trachestoma valve. It is also contemplated that the design of button 10 may be modified to positively engage an insertion limiting protrusion extending outward from locking device 30 to positively situate device 30 in position within aperture 18.

Locking device 30 may also be fabricated using a resilient yet flexible metal or plastic wound in a spring-like shape to recreate the external contours of cylindrical portion 32 and protrusion 34 in a custom spring design.

As a patient's stoma naturally enlarges during the day when button 10 is installed, the patient may use more than one sized locking device 30 during the day such as installing a locking device 30 in the afternoon that has a slightly larger diameter for cylindrical portion 32. When the next morning comes, and the stoma has contracted slightly during the night, a slightly smaller diameter locking device 30 is used.

Another application of locking device 30 arises when a patient desires an enlargement of the stoma. A stoma may be gradually enlarged by the insertion of gradually larger diameter locking devices 30 wherein the diameter of cylindrical portion 32 is increased in small incremental steps, such as 0.010″ to 0.020″ stepped diameter enlargements.

While the invention has been illustrated and described in detail in the drawings and foregoing description of the preferred embodiment, the same is to be considered as illustrative and not restrictive in character, it being understood that only the preferred embodiment has been shown and described and that all changes and modifications that come within the spirit of the invention are desired to be protected.

What is claimed is:

1. A locking device for use with an endostomal tracheostoma button, wherein the endostomal tracheostoma button is fabricated from a resilient flexible material and includes a hollow cylindrical portion disposed into a patient's stoma, and wherein the tracheostoma button includes a lip situated at one end of the cylindrical portion that serves to retain the tracheostoma button within the stoma aperture, the locking device comprising:
a hollow cylinder, said hollow cylinder having a first end and a second end, and wherein the radius of said hollow cylinder is greater than the internal radius of the hollow cylindrical portion of the endostomal tracheostoma button; and
means attached to said first end of said hollow cylinder for limiting insertion depth when said second end of said hollow cylinder is axially inserted into the hollow cylindrical portion of the endostomal tracheostoma button, said means for limiting positioning said second end of said hollow cylinder radially adjacent the lip of the tracheostoma button when said hollow cylinder is fully inserted into the hollow cylindrical portion of the tracheostoma button, and wherein said second end of said hollow cylinder provides outward radial support for the lip of the tracheostoma button.

2. The device of claim 1 wherein said means attached to said first end is a radial protrusion extending radially outward from said hollow cylinder.

3. The device of claim 2 wherein said radial protrusion has a triangular cross-section.

4. A locking device for use with an endostomal tracheostoma button, wherein the endostomal tracheostoma button is fabricated from a resilient flexible material and includes a hollow cylindrical portion insertable into a patient's stoma, and wherein the tracheostoma button includes a radially outwardly extending lip situated at one end of the cylindrical portion that serves to retain the tracheostoma button within the stoma aperture, the locking device comprising:
a hollow cylindrical tube having a first end and a second end, said cylindrical tube having a diameter slightly larger than the inner diameter of the hollow cylindrical portion of the tracheostoma tube; and
an insertion stop member attached to said first end of said cylindrical member, said insertion stop member extending radially outward from said first end of said hollow cylindrical tube, wherein said insertion stop member contacts the hollow cylindrical portion of the tracheostoma tube when the second end of said hollow cylindrical tube is inserted in the hollow cylindrical portion of the trcheostoma button and situated radially adjacent the lip of the tracheostoma button to provide outward radial support to the lip.

5. The device of claim 4 wherein said insertion stop member extends radially outward from and surrounds the cylindrical periphery of said hollow cylindrical portion.

6. A locking device for use with an endostomal tracheostoma button, wherein the endostomal tracheostoma button is fabricated from a resilient flexible material and includes a hollow cylindrical portion insertable into a patient's stoma, and wherein the tracheostoma tube also includes a radially outwardly extending lip situated at the insertion end of the cylindrical portion that retains the tracheostoma button within the stoma aperture, the locking device comprising:
a hollow cylindrical tube having a first end and a second end, said cylindrical tube having a diameter slightly larger than the inner diameter of the hollow cylindrical portion of the tracheostoma tube; and
means attached to said first end of said hollow cylindrical tube for limiting insertion depth when said hollow cylindrical tube is inserted into the hollow cylindrical portion of the endostomal tracheostoma tube so that said second end of said cylindrical tube is situated radially adjacent the lip of the tracheostoma button to provide outward radial support to the lip.

7. The device of claim 6 wherein said means attached to said first end is a radial protrusion extending radially outward from and surrounding the cylindrical periphery of said hollow cylindrical tube.
